(19) United States
(12) Patent Application Publication
(10) Pub. No.: US 2024/0117342 A1
(43) Pub. Date: Apr. 11, 2024

(54) EMBRYONIC CHROMOSOME SIGNAL LIBRARY CONSTRUCTION METHOD, DETECTION METHOD, AND DETECTION SYSTEM THEREOF

(71) Applicant: NATIONAL CENTRAL UNIVERSITY, TAOYUAN CITY (TW)

(72) Inventors: LI-JEN SU, TAOYUAN CITY (TW); SHAO-PING WENG, TAIPEI CITY (TW); YU-YU YEN, TAIPEI CITY (TW); LI-CHING WU, TAOYUAN CITY (TW); HUI-YIN CHIU, TAOYUAN CITY (TW); JUI-HUNG KAO, TAIPEI CITY (TW)

(21) Appl. No.: 18/479,198

(22) Filed: Oct. 2, 2023

(30) Foreign Application Priority Data
Oct. 3, 2022 (TW) 111137584

(51) Int. Cl. C12N 15/10

(52) U.S. Cl. CPC C12N 15/1089

(57) ABSTRACT

An construction method of an embryonic chromosome signal library is provided. The construction method comprises obtaining an embryo and performing whole-genome amplification and next-generation sequencing to obtain a first chromosome signal; mapping the first chromosome signal to a chromosome reference signal to obtain a second chromosome signal; dividing the second chromosome signal within a predetermined interval range to obtain a third chromosome signal; and performing a regression correction on the sequencing read count (RC) of the third chromosome signal to obtain an embryonic chromosome signal library. Furthermore, a detection method and system of embryonic chromosomes are also provided. Thereby, the information comparison of the embryo chromosome signal library is used to determine whether the pre-implantation embryo is abnormal or not to achieve pre-implantation chromosome screening of pre-implantation embryos.

### CROSS-REFERENCE TO RELATED APPLICATION

This application claims the priority benefit of Taiwan application serial no. 111137584, filed 2022 Oct. 3, the full disclosure of which is incorporated herein by reference.

### BACKGROUND

### Technical Field

The disclosure relates to a human pre-implantation genetic screening, particularly a method and system for detecting aneuploidy in embryos.

### Description of Related Art

In general, if a couple has regular sexual intercourse without using contraception and remains unable to conceive after one year, it is referred to as infertility. In vitro fertilization (IVF), also known as artificial fertilization, is a common method used to assist in the treatment of infertility. In IVF, eggs and sperm are extracted, fertilization occurs outside the body, resulting in the development of embryos, which are then implanted back into the mother's body.

However, the success rate of artificial fertilization remains quite low, with only 35-40% of IVF cycles resulting in live births. Chromosomal aneuploidy in blastocysts is the main cause of artificial fertilization failure. If a cell contains 2n chromosomes, it is termed euploid. If a cell has one missing or more chromosomes, it is called aneuploid. When the copy number of chromosomes is greater than 2 or less than 2, it can lead to trisomy or monosomy. Chromosomal aneuploidy is primarily due to errors in oocyte meiosis, which often leads to subsequent embryo implantation failure, miscarriage and even the birth of infants with abnormal chromosomes. The most common chromosomal abnormalities involve trisomy of chromosome 21 (Down syndrome), trisomy of chromosome 13 (Patau syndrome), trisomy of chromosome 18 (Edwards syndrome), and Turner syndrome (45, X), among others.

Therefore, the urgent clinical goal is to find a way to examine embryonic chromosomes for abnormalities before implantation into the mother's body. This is to avoid situations where implantation fails, or if successful, it leads to difficulties in pregnancy and the possibility of giving birth to fetuses with genetic defects.

### SUMMARY

In one aspect, this invention provides an construction method of an embryonic chromosome signal library. The construction method comprises obtaining an embryo and performing whole-genome amplification and next-generation sequencing to obtain a first chromosome signal; mapping the first chromosome signal to a chromosome reference signal to obtain a second chromosome signal; dividing the second chromosome signal within a predetermined interval range to obtain a third chromosome signal; and performing a regression correction on the sequencing read count (RC) of the third chromosome signal to obtain an embryonic chromosome signal library.

According to an embodiment of this construction method of an embryonic chromosome signal library, the embryo is a blastocyst embryo.

According to an embodiment of this construction method of an embryonic chromosome signal library, the chromosome reference signal is a human reference genome sequence (hg19), and the first chromosome signal is obtained by mapping using a sequence alignment tool bowtie2 with the chromosome reference signal, followed by sorting using SAMtools to obtain the second chromosome signal.

According to an embodiment of this construction method of an embryonic chromosome signal library, the predetermined interval range is 1 Mb, and the second chromosome signal is analyzed using Bedtools to obtain the third chromosome signal, which comprises sequencing read count (RC).

According to an embodiment of this construction method of an embryonic chromosome signal library, the regression correction comprises using local regression (Locally estimated scatterplot smoothing, LOESS) to correct the GC content of the third chromosome signal and employing a row-wise median calculation.

In another aspect, this invention provides a detection method of embryonic chromosomes. The detection method comprises using the construction method above to obtain an embryonic chromosome signal library for a test embryo and an embryonic chromosome signal library for a reference embryo; setting a threshold range based on the embryonic chromosome signal library for the reference embryo; and comparing the embryonic chromosome signal library for the test embryo and the embryonic chromosome signal library for the reference embryo to determine a status of the chromosomes in the test embryo. When a signal of a chromosome number in the embryonic chromosome signal library for the test embryo exceeds a default proportion of the threshold range, a status of the chromosome number is determined to be abnormal.

According to an embodiment of the detection method of embryonic chromosomes, the threshold range is defined using quartiles, with a first quartile (Q1) representing the lower limit and a third quartile (Q3) representing the upper limit of the threshold range.

According to an embodiment of the detection method of embryonic chromosomes, the default proportion is set at 70%.

According to an embodiment of the detection method of embryonic chromosomes, if the signal of the chromosome number exceeds 70% of the upper limit of the threshold range, the chromosome number is deemed to have a chromosome gain; if the signal of the chromosome number exceeds 70% of the lower limit of the threshold range, the chromosome number is deemed to have a chromosome deletion.

According to an embodiment of the detection method of embryonic chromosomes, the reference embryo comprises live birth embryos or successfully implanted embryos.

In yet another aspect, this invention provides a detection system of embryonic chromosomes. The detection system comprises a database for storing an embryonic chromosome signal library for a reference embryo obtained using the construction method above; a signal receiving module for receiving an embryonic chromosome signal library for a test embryo obtained using the construction method above; and a comparative analysis module for comparing the embryonic chromosome signal library for the test embryo with the embryonic chromosome signal library for the reference embryo to generate an analysis report. When a signal of a chromosome number in the embryonic chromosome signal library for the test embryo exceeds a default proportion of the threshold range, a status of the chromosome number is determined to be abnormal and is presented in the analysis report.

According to an embodiment of the detection system of embryonic chromosomes, the threshold range is defined using quartiles, with a first quartile (Q1) representing the lower limit and a third quartile (Q3) representing the upper limit of the threshold range.

According to an embodiment of the detection system of embryonic chromosomes, the reference embryo comprises live birth embryos or successfully implanted embryos.

Thereby, this invention can detect whether the pre-implantation embryo is abnormal or not and realize pre-implantation chromosomal screening of the pre-implantation embryo to avoid giving birth to a fetus with genetic defects. The financial and psychological burden of long-term treatment on infertility patients is also reduced.

### BRIEF DESCRIPTION OF THE DRAWINGS

FIG. 1 is a flow chart of the construction method of the embryo chromosome signal library according to an embodiment of this invention.

FIG. 2 is a flowchart illustrating a detection method of embryonic chromosomes according to an embodiment of this invention.

FIG. 3 is a diagram showing the detection results of a test embryo (euploid), with live birth embryos as reference embryos, according to another embodiment of this invention.

FIG. 4 is a diagram showing the detection results of a test embryo (aneuploid), with live birth embryos as reference embryos, according to another embodiment of this invention.

FIG. 5 is a diagram showing the detection results of a test embryo (euploid), with successfully implanted embryos as reference embryos, according to yet another embodiment of this invention.

FIG. 6 is an architectural diagram of a detection system of embryonic chromosomes according to an embodiment of this invention.

### DETAILED DESCRIPTION

The detailed description and technical content of this invention are described below with reference to the drawings. However, the attached drawings are only provided for reference and illustration to help understand this invention and are not intended to limit the scope of this invention.

It should be understood that although terms such as “first”, “second” and “third” may be used herein to describe various components or signals, these components or signals should not be limited by these terms. These terms are mainly used to distinguish one component from another component, or one signal from another signal. In addition, the term “or” used in this disclosure shall, depending on the actual situation, include any one or a combination of more of the associated listed items.

During the in vitro fertilization process, eggs are retrieved from mature follicles within the mother's ovaries. Fertilization is achieved by injecting a single sperm into the egg or by mixing eggs and sperm in a culture dish. The fertilized egg (embryo) is then transferred to the uterus.

Preimplantation Genetic Screening (PGS) is a chromosome count examination conducted before the implantation of embryos during artificial fertilization treatment. This invention is an application of PGS. Furthermore, the analysis algorithms used in this invention are based on the R and Python programming languages.

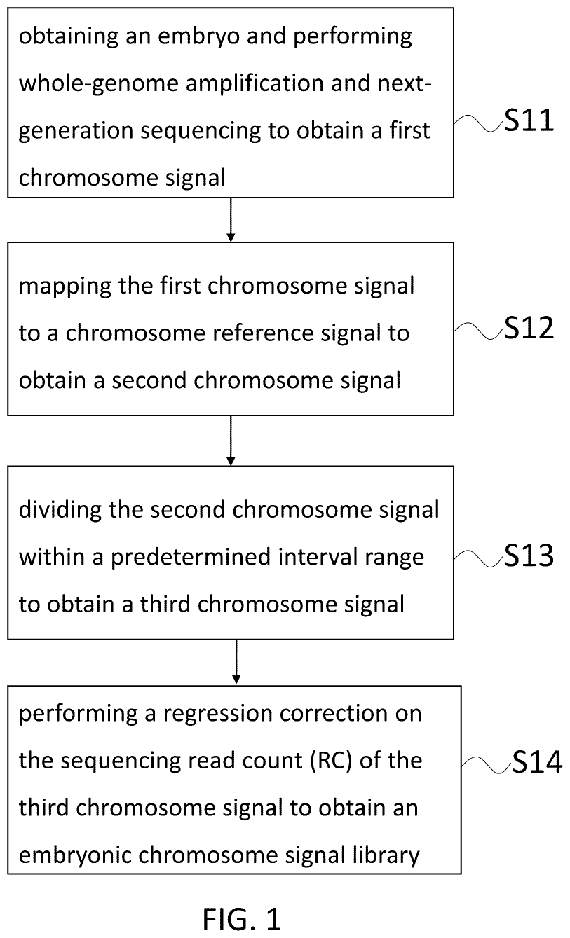

Please refer to FIG. 1, which is a flow chart of the construction method of the embryo chromosome signal library according to an embodiment of this invention. The construction method of the embryonic chromosome signal library of this invention comprises: obtaining an embryo and performing whole-genome amplification and next-generation sequencing to obtain a first chromosome signal S11; mapping the first chromosome signal to a chromosome reference signal to obtain a second chromosome signal S12; dividing the second chromosome signal within a predetermined interval range to obtain a third chromosome signal S13; and performing a regression correction on the sequencing read count (RC) of the third chromosome signal to obtain an embryonic chromosome signal library S14.

In current PGS protocols, embryo biopsy is performed at the cleavage stage on the third day, followed by testing using fluorescence in situ hybridization (FISH). This is a PGS protocol with high acceptance among women with advanced maternal age. However, performing embryo biopsy on the third day during the cleavage stage can potentially harm the implantation potential, which can reduce the live-birth rate in women with advanced maternal age. Therefore, in an embodiment of this invention, the embryos used are blastocyst-stage embryos on the fifth day (blastocyst biopsy), which are less likely to have embryo damage issues.

The following provides a detailed explanation of the process for constructing the embryonic chromosome signal library according to an embodiment of this invention.

In step S11: An embryo is obtained, and whole-genome amplification and next-generation sequencing is performed to obtain a first chromosome signal. In this step, this invention does not specifically limit the method of cell lysis, whole-genome amplification, and next-generation sequencing after obtaining the embryos.

Regarding the method of embryo cell lysis, any method that can thoroughly lyse the embryo cells is suitable. For example, mechanical methods such as high-pressure homogenization, ultrasonication, shaking bead disruption, fine grinding by high-speed bead agitation, or non-mechanical methods such as osmotic shock and chemical treatment can be employed.

Regarding the method of whole-genome amplification and next-generation sequencing, one with ordinary skills in the art can choose different whole-genome sequencing methods based on the specific protocol using the selected genome sequencing technology. For example, any commercially available reagent kits such as Ion Reproseq (Thermo Fisher), PicoPlex (Rubicon Genomics), or Repli-G (Qiagen) can be used for whole-genome amplification. The next-generation sequencing method can also be performed on any high-throughput next-generation sequencing platform, such as IonTorrent, BGlseq, or MGlseq sequencing platforms, among others. Methods provided by manufacturers of sequencing instruments, such as methods provided by Illumina, can also be used. For example, it may refer to methods provided by Illumina's Multiplexing Sample Preparation Guide (Part#1005361; February 2010) or Paired-End Sample Prep Guide (Part#1005063; February 2010) and incorporating them into this invention.

In one embodiment, step S11 can involve using the VeriSeq PGS Kit (Illumina) for whole-genome amplification and next-generation sequencing of embryo cells (Illumina MiSeq System, Illumina, San Diego, CA). After sequencing is complete, the raw data (FASTQ sequence files) representing the first chromosome signal are obtained.

In step S12: The first chromosome signal is then aligned with the human reference genome sequence (hg19), which serves as the chromosome reference signal, using the sequence alignment tool Bowtie2 (v2.5.0). This alignment process generates SAM (Sequence Alignment/Map) formatted alignment files. Subsequently, SAMtools is used to sort and create a Binary Alignment Map (BAM), which represents the second chromosome signal. This process comprises the conversion of SAM to BAM format using SAMtools view, sorting of the BAM files using SAMtools sort, and the creation of an index for the BAM files using SAMtools index.

In step S13: The second chromosome signal is analyzed using Bedtools, where each predetermined interval range is divided into windows, which represents the third chromosome signal. In one embodiment of this invention, the interval range is set to 1 Mb (1,000,000 base pairs), resulting in 3114 windows. The inventors have found that setting the interval range to 1 Mb is advantageous for subsequent data analysis, as it improves analysis efficiency, reduces costs, and enhances the efficiency of determining chromosomal aneuploidy in embryos while lowering the associated expenses.

The third chromosome signal comprises information such as chromosome number, the start point of the interval, the endpoint of the interval, read count (RC), the number of base pairs, the interval size, average coverage depth, or combinations thereof. Specifically, the third chromosome signal must include at least the read count (RC).

In step 14: The third chromosome signal's read count is subjected to regression correction using a local regression (LOESS) method to correct the GC content of the read count. The specific formula for this correction process is provided as follows:

local regression (LOESS):

$$R {C}_{Corr .} = R {C}_{raw} \times \frac{\overline{R C}}{R {C}_{GC , Pred .}}$$

R̅C̅=meanₒᵥₑᵣₐₗₗ
RC_{Corr.}: The read count after LOESS correction.
RC_{raw}: The read count for each predetermined interval range.
RC_{GC,Pred.}: The read count after GC correction following next-generation sequencing.
meanₒᵥₑᵣₐₗₗ: The mean of RC_{raw}.

Next, the RC_{Corr} values are processed using a row-wise median calculation to obtain the corresponding median values (RC_{med}) to obtain an embryonic chromosome signal library.

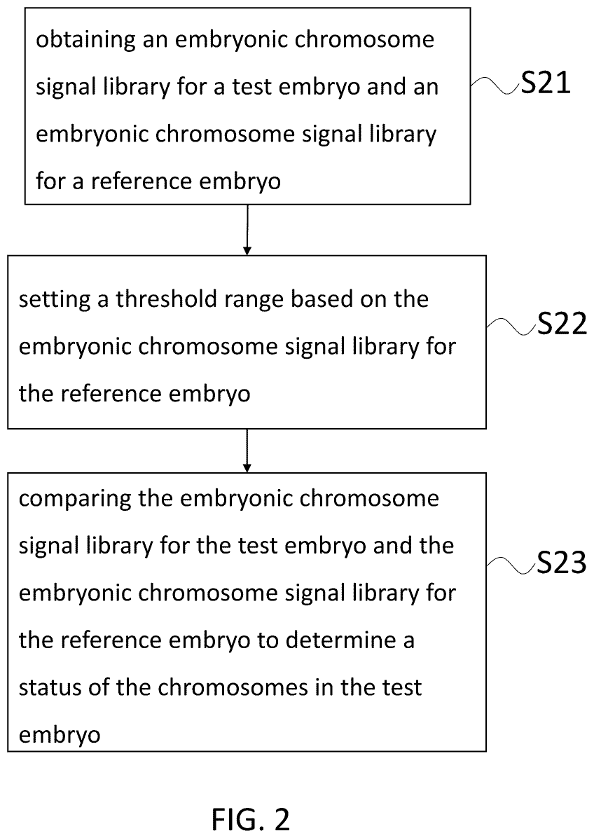

After establishing the embryonic chromosome signal library, the invention provides a method for testing embryo chromosomes using this library. Please refer to FIG. 2. FIG. 2 is a flowchart illustrating a detection method of embryonic chromosomes according to an embodiment of this invention. The detection method of embryonic chromosomes comprises obtaining an embryonic chromosome signal library for a test embryo and an embryonic chromosome signal library for a reference embryo S21; setting a threshold range based on the embryonic chromosome signal library for the reference embryo S22; and comparing the embryonic chromosome signal library for the test embryo and the embryonic chromosome signal library for the reference embryo to determine a status of the chromosomes in the test embryo S23. When a signal of a chromosome number in the embryonic chromosome signal library for the test embryo exceeds a default proportion of the threshold range, a status of the chromosome number is determined to be abnormal.

In one embodiment, the calculation formula for comparing the embryo chromosome signal library of the test embryo and the embryo chromosome signal library of the reference embryo is as follows:

$$R {C}_{final} = 2 \times ( \frac{R {C}_{med - test}}{R {C}_{med - ref}} ) \times ( \frac{{reads}_{baseline}}{{reads}_{mapped}} )$$

RC_{final}: The final read count after the calculation.
RC_{med-test}: The chromosome signal library of the test embryo.
RC_{med-ref}: The chromosome signal library of the reference embryo.
reads_{baseline}: 1,000,000.
reads_{mapped}: 833,767.

It is further explained that the reference embryo's chromosome signal library's threshold range is defined using quartiles. The first quartile (Q1) represents the lower limit of the threshold range, and the third quartile (Q3) represents the upper limit of the threshold range. If the signal of a chromosome number in the test embryo's chromosome signal library exceeds a default proportion of this threshold range, then the status of that chromosome number is determined to be abnormal (aneuploid). Conversely, if the signal from the test embryo's chromosome signal library falls within the default proportion of the threshold range, it is considered normal (euploid). The default proportion may be 70%, for example.

In addition, if the chromosome numbering signal exceeds 70% of the upper limit of the threshold range, it is deemed that the chromosome number has a chromosome increase (such as triploidy). If the chromosome number signal exceeds 70% of the lower limit of the threshold interval, it is considered that the chromosome number has a chromosome deletion (such as a monosomy).

In one embodiment, the reference embryo comprises live birth embryos or successfully implanted embryos.

An example using live birth embryos as the reference embryos is as follows:

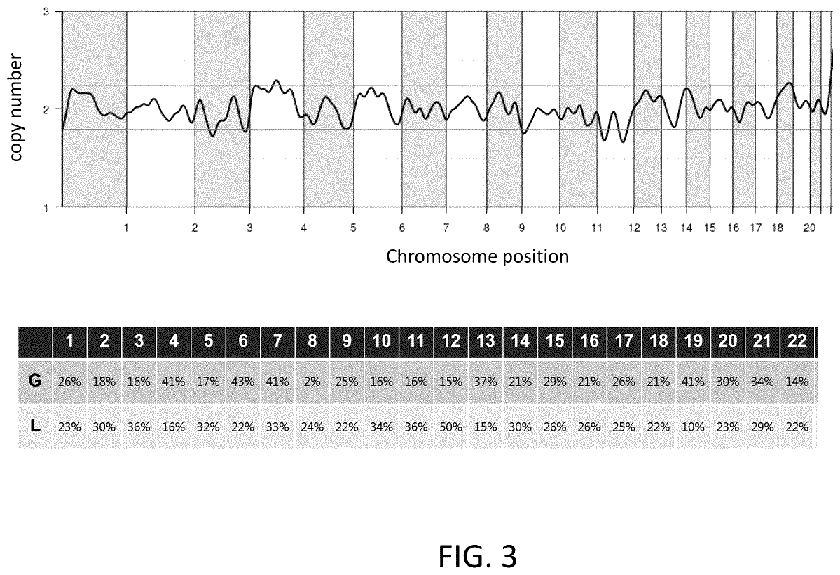

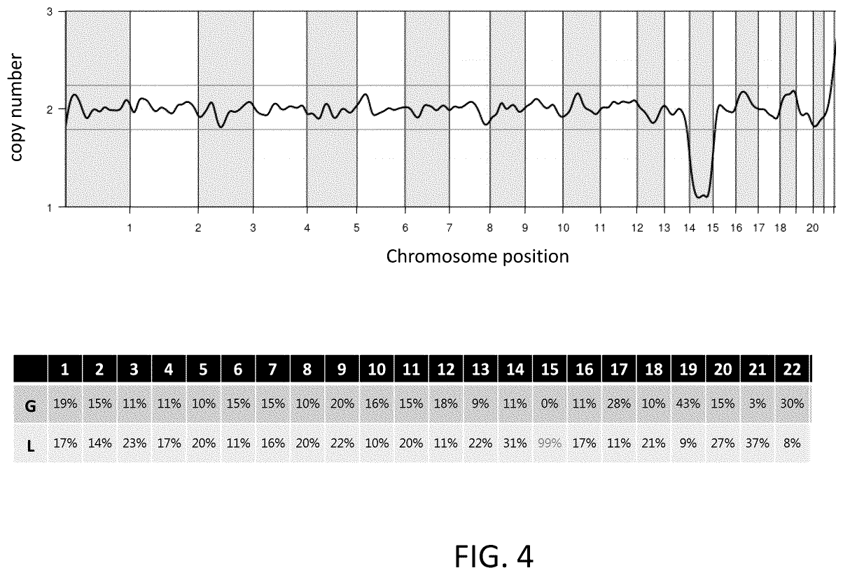

In this example, data from library containing 55 live birth embryo chromosome signals was utilized as reference data. The first quartile (Q1) was defined as 1.79, and the third quartile (Q3) was defined as 2.24. Consequently, the threshold range falls between 1.79 and 2.24 (please refer to the two horizontal lines marked in the upper part of FIGS. 3 and 4).

Please refer to FIGS. 3 and 4, which show the detection results for the test embryos. In FIG. 3, all chromosome signals from the test embryo fall within the default proportion of the threshold range, indicating that it is euploid. In FIG. 4, for the test embryo's 15th chromosome, there are a total of 83 signal values (RC_{final}) after calculation. Therefore, considering a default proportion of 70%, if there are more than 58 signal values outside the threshold range (83*70%=58), it is considered abnormal. In this case, 82 signal values exceeded the lower threshold of 1.79 (99%), which is more than 70%, indicating an abnormality. This implies a deletion in the 15th chromosome of this test embryo, and it should be excluded from further implantation. In the figures, “G” (GAIN) at the bottom represents the proportion of chromosome number signals in the embryo's chromosome signal library exceeding the upper limit of the threshold range, while “L” (LOSS) represents the proportion of chromosome number signals exceeding the lower limit of the threshold range.

Table 1 shows the results of testing 296 embryos using the model established with the reference embryos from live births, as described above. In this experiment, there were 191 cases of successful embryo implantation leading to pregnancy and 105 cases of miscarriage.

TABLE 1

| | | |
|---|---|---|
| True Positive, TP | 121 | |
| False Positive, FP | 68 | |
| False Negative, FN | 70 | |
| True Negative, TN | 37 | |
| Accuracy | 53.4% | Indicates the efficiency of using the |
| [(TP + TN)/(TP + FP + | | detection method of this invention |
| FN + TN)]*100% | | |
| Precision | 64.0% | Pregnancy prediction success rate |
| [TP/(TP + FP)]*100% | | |
| Sensitivity | 63.4% | true positive rate |
| [(TP/(TP + FN)]*100% | | |
| Specificity | 35.2% | true negative rate |
| [(TN/(TN + FP)]*100% | | |

An example of using successfully implanted embryos as reference embryos is as follows:

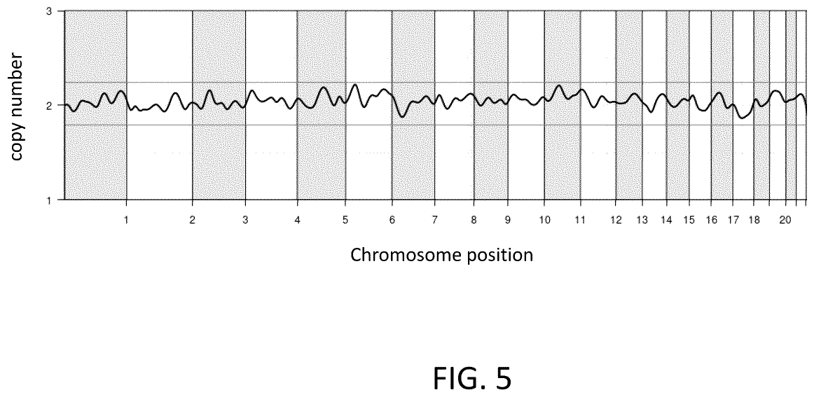

In this embodiment, collected data from 100 embryos that successfully implanted were used as reference data. The defined threshold values for the first quartile (Q1) and third quartile (Q3) were 1.62 and 2.07, respectively. Therefore, the threshold range is defined as between 1.62 and 2.07. In FIG. 5, the chromosome signals of the test embryos all fall within the default proportion in the threshold range, and they are determined to be euploid.

Table 2 provides the results of testing 296 embryos using the model established with reference embryos from successfully implanted embryos. In this experiment, there were 191 cases of successful pregnancy and 105 cases of miscarriage.

TABLE 2

| | | |
|---|---|---|
| True Positive, TP | 159 | |
| False Positive, FP | 82 | |
| False Negative, FN | 32 | |
| True Negative, TN | 23 | |
| Accuracy | 61.5% | Indicates the efficiency of using the |
| [(TP + TN)/(TP + FP + | | detection method of this invention |
| FN + TN)]*100% | | |
| Precision | 66.0% | Pregnancy prediction success rate |
| [TP/(TP + FP)]*100% | | |
| Sensitivity | 83.2% | true positive rate |
| [(TP/(TP + FN)]*100% | | |
| Specificity | 21.9% | true negative rate |
| [(TN/(TN + FP)]*100% | | |

The results from the two embodiments above demonstrate that, regardless of whether the reference embryos are derived from the chromosomal signal profiles of live birth embryos or successfully implanted embryos, using the method for establishing the embryo chromosomal signal library of this invention and employing the completed database for embryo testing can predict a pregnancy success rate of over 60%.

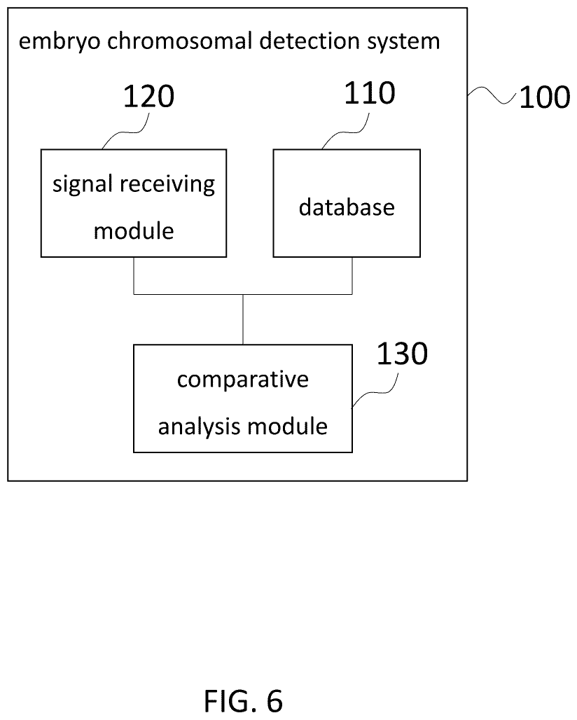

Please refer to FIG. 6, which is an architectural diagram of a detection system of embryonic chromosomes according to an embodiment of this invention. The embryo chromosomal detection system 100 of this invention comprises a database 110 for storing an embryonic chromosome signal library of a reference embryo; a signal reception module 120 for receiving an embryonic chromosomal signal library of a test embryo; and a comparative analysis module 130 for comparing the embryonic chromosome signal library of the test embryo with the embryonic chromosome signal library of the reference embryo to generate an analysis report. If the signal of a particular chromosome number in the chromosomal signal library of the test embryo exceeds a default proportion of a threshold range in the chromosomal signal profile of the reference embryos, it is determined that the status of that chromosome number is abnormal and is presented in the analysis report.

In one embodiment, the reference embryo's embryonic chromosome signal library and the test embryo's embryonic chromosome signal library are obtained using the construction method of the embryonic chromosome signal database, as described above.

In one embodiment, the threshold range is defined using quartiles, with a first quartile (Q1) representing the lower limit and a third quartile (Q3) representing the upper limit of the threshold range. The default proportion may be 70%.

In one embodiment, if the signal of the chromosome number exceeds 70% of the upper limit of the threshold range, the chromosome number is deemed to have a chromosome gain; if the signal of the chromosome number exceeds 70% of the lower limit of the threshold range, the chromosome number is deemed to have a chromosome deletion.

In one embodiment, the reference embryo comprises live birth embryos or successfully implanted embryos.

In one embodiment, the analysis report can be presented on the screen display interface of the embryonic chromosome detection system of this invention. It can also be output through an output module as a hardcopy document or transmitted to a user's electronic device, such as a phone, tablet, desktop computer, or laptop, etc.

In summary, the methods and systems for constructing embryonic chromosome signal databases provided by this invention, and their utilization, enable the definition of reference threshold ranges for reference embryos (such as live birth embryos or successfully implanted embryos) based on a substantial amount of clinical test data. These thresholds are then applied to the detection of the target embryos to assist in clinical pre-implantation embryo anomaly assessment. This allows for the identification of aneuploid embryo cells and provides clinical guidance, ultimately realizing pre-implantation chromosome screening before embryo implantation.

The above description represents preferred and workable embodiments of this invention, but this shall not be considered as limiting the scope of the patent. Any structural variations or equivalent modifications within the scope of this invention that are derived from the content of this specification and the accompanying drawings are also considered to be part of this invention.

1. An construction method of an embryonic chromosome signal library, comprising:
obtaining an embryo and performing whole-genome amplification and next-generation sequencing to obtain a first chromosome signal;
mapping the first chromosome signal to a chromosome reference signal to obtain a second chromosome signal;
dividing the second chromosome signal within a predetermined interval range to obtain a third chromosome signal; and
performing a regression correction on the sequencing read count (RC) of the third chromosome signal to obtain an embryonic chromosome signal library.

2. The construction method of an embryonic chromosome signal library of claim 1, wherein the embryo is a blastocyst biopsy embryo.

3. The construction method of an embryonic chromosome signal library of claim 1, wherein the chromosome reference signal is a human reference genome sequence (hg19), and the first chromosome signal is obtained by mapping using a sequence alignment tool bowtie2 with the chromosome reference signal, followed by sorting using SAMtools to obtain the second chromosome signal.

4. The construction method of an embryonic chromosome signal library of claim 3, wherein the predetermined interval range is 1 Mb, and the second chromosome signal is analyzed using Bedtools to obtain the third chromosome signal, which comprises sequencing read count (RC).

5. The construction method of an embryonic chromosome signal library of claim 4, wherein the regression correction comprises using local regression (LOESS) to correct the GC content of the third chromosome signal and employing a row-wise median calculation.

6. A detection method of embryonic chromosomes, comprising:
using the construction method of claims 1 to obtain an embryonic chromosome signal library for a test embryo and an embryonic chromosome signal library for a reference embryo;
setting a threshold range based on the embryonic chromosome signal library for the reference embryo; and
comparing the embryonic chromosome signal library for the test embryo and the embryonic chromosome signal library for the reference embryo to determine a status of the chromosomes in the test embryo,
when a signal of a chromosome number in the embryonic chromosome signal library for the test embryo exceeds a default proportion of the threshold range, a status of the chromosome number is determined to be abnormal.

7. The detection method of embryonic chromosomes of claim 6, wherein the threshold range is defined using quartiles, with a first quartile (Q1) representing the lower limit and a third quartile (Q3) representing the upper limit of the threshold range.

8. The detection method of embryonic chromosomes of claim 7, wherein the default proportion is set at 70%.

9. The detection method of embryonic chromosomes of claim 8, wherein if the signal of the chromosome number exceeds 70% of the upper limit of the threshold range, the chromosome number is deemed to have a chromosome gain; if the signal of the chromosome number exceeds 70% of the lower limit of the threshold range, the chromosome number is deemed to have a chromosome deletion.

10. The detection method of embryonic chromosomes of claim 6, wherein the reference embryo comprises live birth embryos or successfully implanted embryos.

11. A detection system of embryonic chromosomes, comprising:
a database for storing an embryonic chromosome signal library of a reference embryo obtained using the construction method of claims 1;
a signal receiving module for receiving an embryonic chromosome signal library of a test embryo obtained using the construction method of claims 1; and
a comparative analysis module for comparing the embryonic chromosome signal library of the test embryo with the embryonic chromosome signal library of the reference embryo to generate an analysis report,
when a signal of a chromosome number in the embryonic chromosome signal library for the test embryo exceeds a default proportion of the threshold range, a status of the chromosome number is determined to be abnormal and is presented in the analysis report.

12. The detection system of embryonic chromosomes of claim 11, wherein the threshold range is defined using quartiles, with a first quartile (Q1) representing the lower limit and a third quartile (Q3) representing the upper limit of the threshold range.

13. The detection system of embryonic chromosomes of claim 11, wherein the reference embryo comprises live birth embryos or successfully implanted embryos.
